(19) United States
(12) Patent Application Publication
(10) Pub. No.: US 2020/0015670 A1
(43) Pub. Date: Jan. 16, 2020

(54) DEVICES AND METHODS FOR INTERNAL IMAGING

(71) Applicant: Safeview Medical, LLC, Houston, TX (US)

(72) Inventors: Tarun Mullick, Saint Charles, IL (US); Manish Ahuja, Houston, TX (US); Charles Houssiere, Houston, TX (US); Ashok Gowda, Houston, TX (US); Siva Praneeth Vayugundla, College Station, TX (US)

(21) Appl. No.: 16/336,112

(22) PCT Filed: Sep. 24, 2017
(86) PCT No.: PCT/US17/53133

§ 371 Date: Mar. 24, 2019

Related U.S. Application Data
(60) Provisional application No. 62/398,800, filed on Sep. 23, 2016.

(51) Int. Cl. A61B 1/273; A61B 1/00; A61B 1/005; A61B 1/018; A61B 1/015; A61B 5/145; A61B 5/01; A61B 5/03

(52) U.S. Cl. CPC A61B 1/273; A61B 1/00119; A61B 1/0057; A61B 1/018; A61B 1/015; A61B 5/14507; A61B 1/00133; A61B 5/14539; A61B 5/01; A61B 5/036; A61B 1/00098

(57) ABSTRACT

The invention relates to devices and methods for visualizing and/or interacting with internal body tissues. More particularly, the present invention relates to endoscopic methods and devices for visualizing and/or interacting with the gastrointestinal and/or pancreaticobiliary systems, such as with one use or disposable devices, such as with duodenoscopes. A device for visualizing and/or interacting with internal body tissues may generally include a handpiece, a distal assembly, and/or a connecting conduit. A plurality of conduits and/or channels may span through the connecting conduit from the handpiece to the distal assembly, and may, for example, carry fluid/gas connections, electrical/sensor connections, such as for a camera, mechanical connections and/or carry medical devices through a working channel. The device may also reduce the needs associated with reusable devices such as for reducing risks associated with improper sterilization.

### CROSS-REFERENCE TO RELATED APPLICATIONS

This application is a Patent Cooperation Treaty International Application and claims the benefit and priority of U.S. provisional patent application Ser. No. 62/398,800, filed Sep. 23, 2016, entitled “DEVICES AND METHODS FOR GASTROINTESTINAL IMAGING”, the contents of which is hereby incorporated by reference in its entirety.

### COPYRIGHT NOTICE

A portion of the disclosure of this patent document contains material which is subject to copyright protection. The copyright owner has no objection to the facsimile reproduction by anyone of the patent document or the patent disclosure, as it appears in the U.S. Patent and Trademark Office patent file or records, but otherwise reserves all copyright rights whatsoever.

### FIELD OF THE INVENTION

The invention relates to devices and methods for visualizing and/or interacting with internal body tissues. More particularly, the present invention relates to endoscopic methods and devices for visualizing and/or interacting with the gastrointestinal and/or pancreaticobiliary systems. Further, the present invention relates to one use or at least partially disposable devices for visualizing and/or interacting with the gastrointestinal and/or pancreaticobiliary systems, such as with duodenoscopes.

### BACKGROUND OF THE INVENTION

Endoscopes for medical use have been adopted for various diagnostic and medical treatment procedures. Endoscopes have been used for the diagnosis and treatment of a wide range of diseases and disorders that often require a physician to access the tortuous and relatively small cross-sectional areas of a patient's internal anatomical body lumens. A patient's pancreaticobiliary system (including the anatomical regions of the gall bladder, pancreas, and the biliary tree), for example, is accessed for diagnosis, and/or treatment of disorders of certain portions of the digestive system.

During treatment of the digestive system, endoscopes are often used to access and visualize a patient's pancreaticobiliary system. Once the endoscope is positioned in the desired body portion, a treatment instrument can be advanced through the working channel of the endoscope to the desired body portion. The endoscope and treatment instrument may then be manipulated as desired for visualization and treatment respectively.

Endoscopic retrograde cholangiopancreatography (ERCP) is one example of a medical procedure that uses an endoscope. ERCP enables the physician to diagnose problems in the liver, gallbladder, bile ducts, and pancreas. The liver is a large organ that, among other things, makes a liquid called bile that helps with digestion. The gallbladder is a small, pear-shaped organ that stores bile until it is needed for digestion. The bile ducts are tubes that carry bile from the liver to the gallbladder and small intestine. These ducts are sometimes called the biliary tree. The pancreas is a large gland that produces chemicals that help with digestion and hormones such as insulin.

The biliary system delivers bile produced by the liver to the duodenum where the bile assists other gastric fluids in digesting food. The biliary system includes the liver, as well as a plurality of bodily channels and organs that are disposed between the liver and the duodenum. Within the liver lobules, there are many fine “bile canals” that receive secretions from the hepatic cells. The canals of neighboring lobules unite to form larger ducts, and these converge to become the “hepatic ducts.” They merge, in turn, to form the “common hepatic duct.” The “common bile duct” is formed by the union of the common hepatic and the cystic ducts. It leads to the duodenum, where its exit is guarded by a sphincter muscle. This sphincter normally remains contracted until the bile is needed, so that bile collects in the common bile duct and backs up to the cystic duct. When this happens, the bile flows into the gallbladder and is stored there.

ERCP is used primarily to diagnose and treat conditions of the bile ducts, including gallstones, inflammatory strictures, leaks (from trauma and surgery), and cancer. ERCP combines the use of x-rays and an endoscope. Through the endoscope, the physician can see the inside of the stomach and duodenum, and inject dyes into the ducts in the biliary tree and pancreas so they can be seen on x-rays.

An ERCP is performed primarily to identify and/or correct a problem in the bile ducts or pancreas. For example, if a gallstone is found during the exam, it can often be removed by means of a treatment instrument, eliminating the need for major surgery. If a blockage in the bile duct causes yellow jaundice or pain, it can be relieved through the use of a treatment instrument inserted through the endoscope.

Recent attention has been directed to cases of patient illness due to contamination and improper sterilization of reusable endoscopes such as duodenoscopes. High infection rates secondary to current reusable duodenoscopes have sparked significant problems for patients, hospitals, and doctors. Various methods of reprocessing and additional steps within the reprocessing have been implemented. Currently this is a major problem in medicine related to infections and deaths that should be avoidable.

### SUMMARY OF THE INVENTION

The invention relates to devices and methods for visualizing and/or interacting with internal body tissues. More particularly, the present invention relates to endoscopic methods and devices for visualizing and/or interacting with the gastrointestinal and/or pancreaticobiliary systems. Further, the present invention relates to one use or disposable devices for visualizing and/or interacting with the gastrointestinal and/or pancreaticobiliary systems, such as with duodenoscopes.

In general, a device for visualizing and/or interacting with internal body tissues may generally include a handpiece, a distal assembly, and/or a connecting conduit. The device may further generally be used to introduce the distal assembly to a location in proximity to an internal body tissue of interest, such as, for example, portions of the gastrointestinal and/or pancreaticobiliary systems, further for example the tubular body structures of those systems. In some exemplary embodiments, a plurality of conduits and/or channels may span through the connecting conduit from the handpiece to the distal assembly, and may, for example, carry fluid/gas connections, electrical/sensor connections, mechanical connections and/or carry medical devices through a working channel. The connecting conduit may generally be flexible and/or deformable and interact with the actions of the pull wires to direct the distal end of the connecting conduit in a desired direction. The device may also be, in general, disposable and/or single use. Disposable or single use devices may be desirable, for example, to aid in reducing the incidence of infection or contamination from improper handling or sterilizing of reusable devices, reducing the need for maintenance, allowing for selection of different materials that do not necessarily require durability against repeated use/sterilization and allowing for lower cost materials. Devices may also utilize modular designs which may include independently replaceable portions, such as disposable portions and reusable portions. Portions may also be designed to be hot-swappable, such as to accommodate replacement of portions, such as due to malfunction or the like, during use.

In one aspect, a device for visualizing and/or interacting with internal body tissues may generally utilize a plurality of mechanical directors for guiding the trajectory of the distal assembly when being inserted and/or navigated through body tubes and/or cavities. In some exemplary embodiments, the mechanical directors may generally include a plurality of pull wires which may pull and/or push on the distal assembly while contained within the connection conduit for altering and/or articulating the direction/trajectory of the distal assembly. A further mechanical director may also be used, for example, to control an elevator and/or other feature for manipulating a medical device at the distal assembly. Mechanical directors may be controlled by manual mechanisms or powered mechanisms, such as motors. Either analog interfaces, digital interfaces or a combination thereof between controls and the mechanical directors may be utilized. With digital interfaces, a variety of different controllers may be utilized, such as controls on the handpiece, wireless controls (e.g. control from a mobile device, tablet, remote control, computer or wireless controller), wired controllers, and/or any other appropriate controller.

In another aspect, the device may generally include a camera in the distal assembly for visualizing body tissues. In some exemplary embodiments, the camera may be side viewing relative to the axis of the insertion of the device. Further, the camera may generally be connected through the connecting conduit and/or handpiece for real time viewing and/or on demand visual capture during a procedure. The camera and/or processing system for the camera may also include, for example, dynamic and/or directional brightness control, such as with a light sensor, panoramic image capture/image stitching, image stabilization and/or other features.

In an exemplary embodiment, a device for imaging a body cavity comprises a handpiece, a connecting conduit extending from said handpiece from a proximal end, a distal assembly connected to a distal end of said connecting conduit, a plurality of controls coupled to said handpiece connected to said distal assembly through a plurality of pull wires extending from mechanical actuators in said handpiece through said connecting conduit to said distal assembly, said plurality of pull wires being adapted to alter the trajectory of said distal assembly, a working channel having an entry point on said handpiece and extending through said connecting conduit to an aperture on said distal assembly, and at least one sensor disposed on said distal assembly in communication with a sensor port on said handpiece.

In an exemplary embodiment, a device for imaging a body cavity comprises a handpiece, a connecting conduit extending from said handpiece from a proximal end, a distal assembly connected to a distal end of said connecting conduit, a plurality of controls digitally coupled to powered actuators in said handpiece connected to said distal assembly through a plurality of pull wires extending from said handpiece through said connecting conduit to said distal assembly, said plurality of pull wires being adapted to alter the trajectory of said distal assembly, a working channel having an entry point on said handpiece and extending through said connecting conduit to an aperture on said distal assembly, and at least one sensor disposed on said distal assembly in communication with a sensor port on said handpiece.

The present invention together with the above and other advantages may best be understood from the following detailed description of the embodiments of the invention and as illustrated in the drawings. The following description, while indicating various embodiments of the invention and numerous specific details thereof, is given by way of illustration and not of limitation. Many substitutions, modifications, additions or rearrangements may be made within the scope of the invention, and the invention includes all such substitutions, modifications, additions or rearrangements.

### BRIEF DESCRIPTION OF THE FIGURES

The drawings accompanying and forming part of this specification are included to depict certain aspects of the invention. A clearer impression of the invention, and of the components and operation of systems provided with the invention, will become more readily apparent by referring to the exemplary, and therefore non-limiting, embodiments illustrated in the drawings, wherein identical reference numerals designate the same components. Note that the features illustrated in the drawings are not necessarily drawn to scale.

FIGS. 1 and 1a illustrate the external features of a device for visualizing and/or interacting with internal body tissues in some exemplary embodiments of the present invention;

FIGS. 1b and 2 illustrate embodiments of the internal conduits and mechanical mechanisms of the device of FIG. 1;

FIGS. 1c and 1d illustrate alternative external features of a device of FIG. 1;

FIGS. 2a and 2b illustrate flexion of a connecting conduit from pushing/pulling pull wires;

FIG. 2c illustrates pinning of a support structure to provide resistance to compression and a return action;

FIG. 3 illustrates an embodiment of a distal assembly of the device of FIGS. 1, 1c and 1d;

FIGS. 4 and 4a illustrate an example of a support structure for a connecting conduit including interconnecting segments;

FIGS. 5 and 5a illustrate an example of a support structure for a connecting conduit including a unitary flexible structure; and

FIGS. 6, 6a, 6b and 6c illustrate an example of powered actuators and reels for the device of FIG. 1, 1c or 1d.

### DETAILED DESCRIPTION OF THE INVENTION

The detailed description set forth below is intended as a description of the presently exemplified methods, devices and systems provided in accordance with aspects of the present invention, and is not intended to represent the only forms in which the present invention may be practiced or utilized. It is to be understood, however, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and systems similar or equivalent to those described herein can be used in the practice or testing of the invention, the exemplified methods, devices and systems are now described.

The invention relates to devices and methods for visualizing and/or interacting with internal body tissues. More particularly, the present invention relates to endoscopic methods and devices for visualizing and/or interacting with the gastrointestinal and/or pancreaticobiliary systems. Further, the present invention relates to one use or at least partially disposable devices for visualizing and/or interacting with the gastrointestinal and/or pancreaticobiliary systems, such as with duodenoscopes.

In general, a device for visualizing and/or interacting with internal body tissues may generally include a handpiece, a distal assembly, and/or a connecting conduit. The device may further generally be used to introduce the distal assembly to a location in proximity to an internal body tissue of interest, such as, for example, portions of the gastrointestinal and/or pancreaticobiliary systems, further for example the tubular body structures of those systems. In some exemplary embodiments, a plurality of conduits and/or channels may span through the connecting conduit from the handpiece to the distal assembly, and may, for example, carry fluid/gas connections, electrical/sensor connections, mechanical connections and/or carry medical devices through a working channel or multiple working channels. Sensing devices, such as cameras, pH sensors, pressure sensors, oxygen sensors, temperature sensors, position/orientation sensors, accelerometers, chemical composition sensors, tissue or fluid analysis sensors, imaging sensors and/or any other appropriate sensing devices may be utilized in the distal assembly to collect data from the body structure being examined and/or about the state of the distal assembly in the body. Transmission of information from sensors in the working channel may, for example, be accomplished through a wired connection carried through the working channel to an external device, such as a signal processor, computer or mobile device, or also through wireless transmission, such as via proprietary signal transmission or standard connectivity, such as WiFi, Ant+ or Bluetooth.

The device may also be, in general, disposable and/or single use. Disposable or single use devices may be desirable, for example, to aid in reducing the incidence of infection or contamination from improper handling or sterilizing of reusable devices, reducing the need for maintenance, allowing for selection of different materials that do not necessarily require durability against repeated use/sterilization and allowing for lower cost materials. With disposable and/or single use devices, the materials and components may be selected for lower cost and/or without requiring higher durability/longevity, such as may be necessary for repeated sterilization and/or other cleaning procedures with multi-use devices.

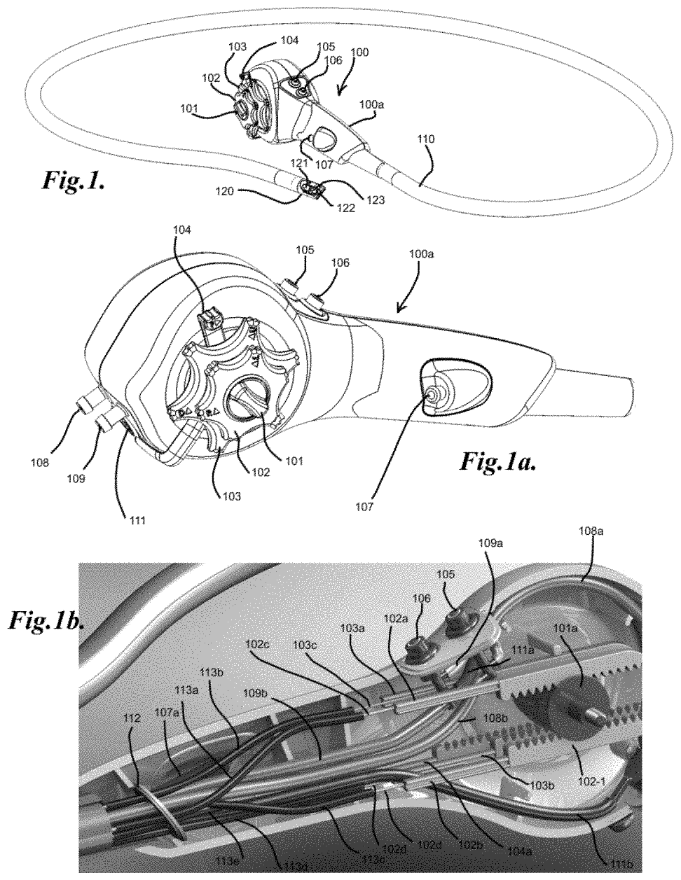

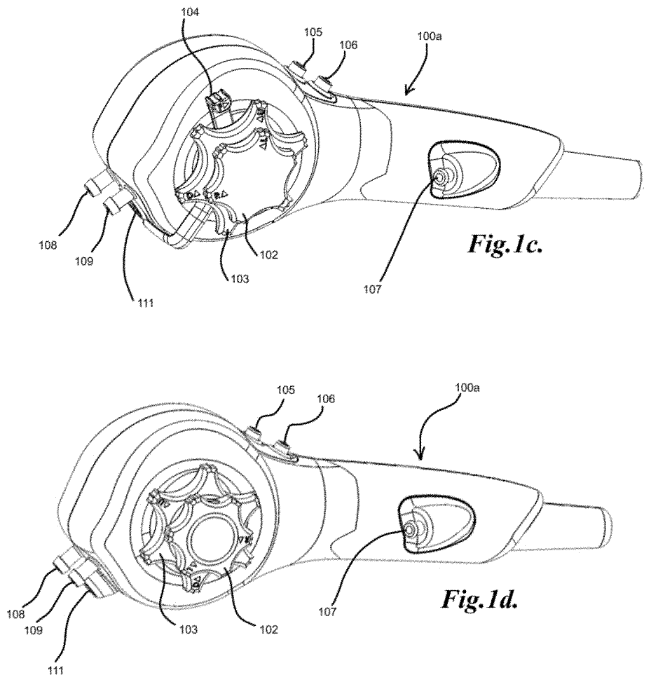

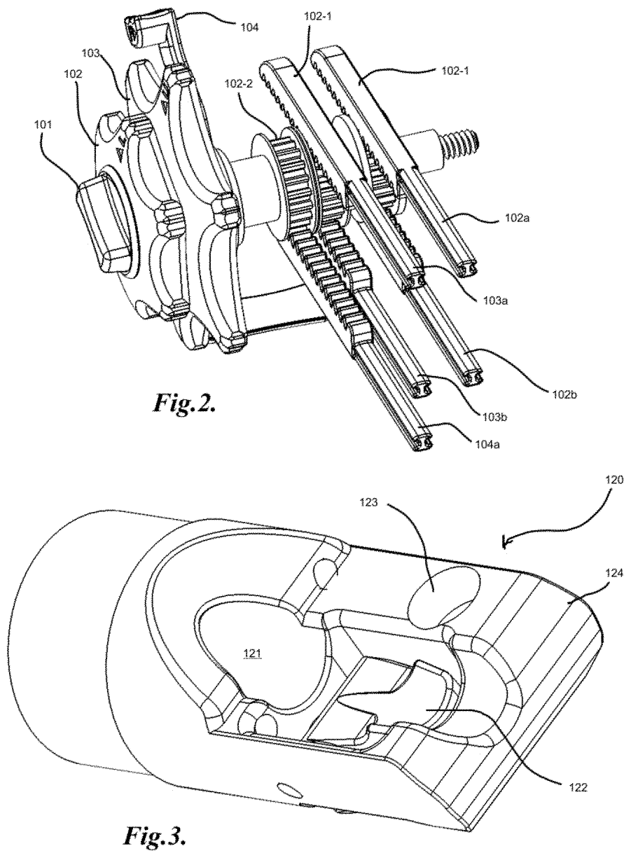

FIGS. 1 and 1a illustrate the exterior of an example of a device 100 for visualizing and/or interacting with internal body tissues with a handpiece body 100a, a plurality of mechanical controls 101, 102, 103, 104, fluid/gas controls 105, 106, a working channel port 107, a connecting conduit 110, a distal assembly 120, a power/water connector 111, air connection 109, and vacuum connection 108.

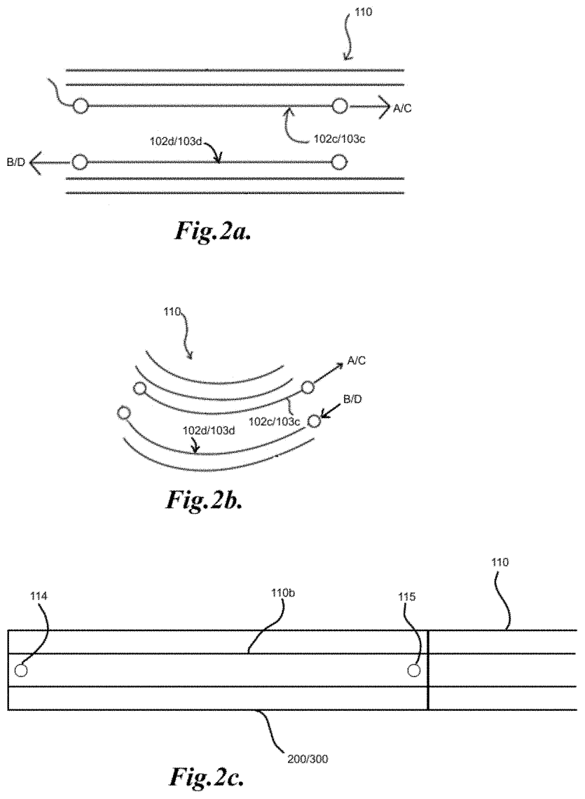

In one aspect, a device for visualizing and/or interacting with internal body tissues may generally utilize a plurality of mechanical directors for guiding the trajectory of the distal assembly when being inserted and/or navigated through body tubes and/or cavities. In some exemplary embodiments, the mechanical directors may generally include a plurality of pull wires which may pull and/or push on the distal assembly while contained within the connection conduit for altering and/or articulating the direction/trajectory of the distal assembly, as illustrated in FIGS. 1b and 2. Pull wires may be made from any appropriate material, such as metal and metal alloys, carbon fibers, fiber glass, polymer strands, natural fibers and/or any other appropriate material or combination thereof. In some exemplary embodiments, control wheels or other actuator controls, such as control wheels 102, 103 as illustrated, may generally articulate pull wires to direct right, left, up and down orienting of the distal assembly, with control wheel 102 directing right and left and control wheel 103 directing up and down, as illustrated. A locking mechanism may also be included to lock the mechanicals in place, as illustrated with locking switch 101. In some embodiments, the locking mechanicals may not be employed, such as where the pull wires remain in a position without locking, as illustrated with the handpiece body 100a in FIGS. 1c and 1d. The control wheels 102, 103 may be mechanically coupled to the actuators or they may be digitally coupled. In digital coupled embodiments, the motion or responsiveness of the control wheels 102, 103 may be adjustable and/or tuned to provide more natural or predictable control for a user. Further, digital controls may be adapted to provide more constant or smooth operation by automatically varying control signals to the actuators, such as due to variability in the mechanical portions of the device 100. With digital interfaces, a variety of different controllers may be utilized, such as controls on the handpiece, wireless controls (e.g. control from a mobile device, tablet, remote control, computer or wireless controller), wired controllers, and/or any other appropriate controller.

In general, as illustrated in FIGS. 2a and 2b, pulling/pushing of a corresponding pair of pull wires, such as direction A/C for pull wires 102c, 102d and/or direction B/D for pull wires 103c, 103d may cause flexion of at least a portion of the connecting conduit 110 as shown in FIG. 2b.

A further mechanical director may also be used, for example, to control an elevator and/or other feature for manipulating a medical device at the distal assembly, as illustrated with elevator actuator 104 controlling elevator 122. The elevator 122 may generally, for example, push on the medical device exiting the working channel aperture 121 to elevate and/or articulate the medical device to a desired location, such as to collect samples and/or place the medical device in proximity with the tissue wall.

In general, any appropriate mechanical actuators may be utilized to control pull wires for altering the direction/trajectory of the distal assembly and/or controlling the elevator. FIGS. 1b and 2 illustrate the use of rack and pinion-like mechanisms for controlling the motion of pull wires, as shown with pinions 102-2 (connected to the control wheels and/or actuators) acting on racks 102-1 coupled to pull wire rods 102a, 103a, 102b, 103b, 104a connected to pull wires 102c, 103c, 102d, 103d, 104b, respectively, which may generally be housed within sheaths 113a, 113b, 113c, 113d, 113e, respectively, for conveyance in the connecting conduit 110. A sheath/conduit guide 112 may also be utilized to arrange the various sheaths and conduits leading into the connecting conduit 110. The connecting conduit 110 may be, in general, flexible and/or compressible/stretchable such that it may deform in response to the pulling/pushing forces of the pull wires to effect the alteration in trajectory/orientation of the distal assembly 120. Elastomeric or otherwise flexible materials may be utilized, or for example, woven materials that may accommodate flexing and compression.

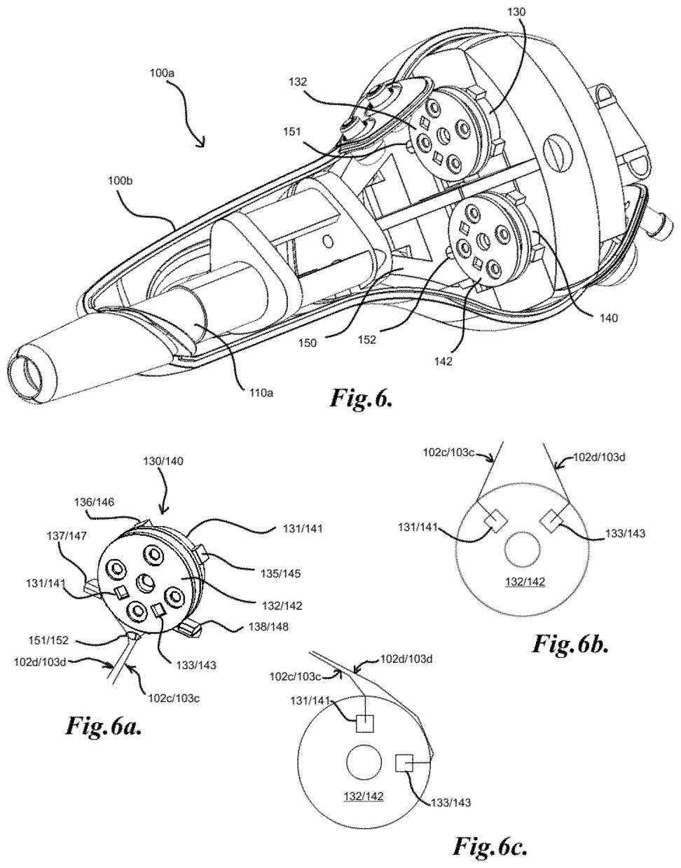

In some embodiments, motorized or powered mechanical actuators may be utilized to control pull wires. FIGS. 6 and 6a illustrate the use of powered actuators, as shown with example motors 130, 140. In some embodiments, the motors 130, 140 may be utilized to wind and dewind pull wires 102c, 102d, 103c, 103d onto and off reels 132, 142 to direct right, left, up and down orienting of the distal assembly by flexion of at least a portion of the connecting conduit 110. The pull wires 102c, 102d, 103c, 103d may further pass into the connecting conduit 110 through a wire guide 150 with entries 151, 152. This may be desirable to aid in guiding the pull wires 102c, 102d, 103c, 103d with the change of direction from the winding/dewinding from the reels 132, 142 to the linear direction along the connecting conduit 110. The reels 132, 142 may also be positioned in other orientations where the directional change is not present. In general, the pull wires 102c, 102d, 103c, 103d may attach to the reels 132, 142, such as at attachment points 131/141, 133/143 as illustrated in FIGS. 6a, 6b and 6c. It may be desirable to position the attachment points 131/141, 133/143 close in an arc, such as less than 90 degrees apart or more particularly less or equal to 45 degrees apart on the arc of the reels 132, 142, such that during rotation of the reels 132, 142, tension is better maintained with less slack in the pull wires 102c, 102d, 103c, 103d, as shown between the rotation in FIGS. 6a and 6b. In some embodiments, the reels 132, 142 may include features for preventing overturning in either or both directions of rotation, such as to prevent damage or overflexion of the connecting conduit 110 during use. In some embodiments, the reels 132, 142 may feature mechanical stops to prevent overturning. In other embodiments, the reels 132, 142 may feature position sensing such that the motors 130, 140 are stopped to prevent overturning. FIG. 6a illustrates an example of position sensing with limit sensors 137/147 and 138/148 detecting the alignment of fiducials 136/146 and 135/145 during the rotation of the reels 132, 142. The position sensing may employ any appropriate sensing or switching technology, such as, for example, magnetic Hall Effect sensors, electric contact switches, optical sensors/switches, tension sensors on the pull wires, physical switches and/or other appropriate position or limit sensors. For example, the fiducials 136/146 and 135/145 may include magnets such that when aligned with the limit sensors 137/147 and 138/148, the magnetic field may trigger a Hall effect sensor in the limit sensors to stop the motors. This may be particularly desirable in digital control systems where there is no direct mechanical coupling between the controls and the motors/reels. Position sensing may also be employed to modulate the speed of the motors in portions of the rotation of the reels 132, 142. For example, due to the shape and reeling/unreeling of the pull wires, there may be certain portions of the rotations where there is additional slack or lack of tension in the pull wires that may generate a “dead zone” or an observable slowing down in the response if the motors remained at a constant speed. The position sensing may be utilized to detect these dead zones to speed up the motors to provide a more constant response during use.

Motors such as gear motors may be utilized to provide the powered rotation of the reels 132, 142. In general, gear motors with high gearing ratios may be utilized such that the high gearing ratio may act as a form of rotation lock when the motor is not on, as this may remove the need for a separate locking mechanism to prevent further rotation or backrotation of the reels 132, 142 when the motors are off. For example, ˜500:1 or higher gear ratio motors may be utilized to generate the locking effect.

In some embodiments, the connecting conduit 110 or portions thereof may be constructed from any appropriate material, such as, for example, medical grade plastic tubing, such as polycarbonate (PC), polyurethane, polyethylene (PE), polypropylene (PP), polylactic acid (PLA), silicone, nylon, polyvinylchloride (PVC), polyethylene terephthalate (PET), polytetrafluoroethylene (PTFE), acrylonitrile butadiene styrene (ABS), polyether sulphone (PES), polyetheretherketone (PEEK), fluorinated ethylene propylene (FEP), other biocompatible polymers, or any combination thereof.

In some exemplary embodiments, the connecting conduit 110 may include a flexible or deformable support structure, such as within an outer sheathing or being integral to a sheathing. The support structure may interact generally with the pull wires to direct the distal assembly 120 of the connecting conduit 110 in a desired direction or orientation, in addition to, for example, providing increased rigidity or resistance to pinching/crushing for a sheathing. In general, the pull wires may be carried in the connecting conduit 110, such as, for example, within a working channel or close to the center of the connecting conduit 110 such that when the pull wires are pulled to cause curvature of the connecting conduit 110 or portion thereof, less slack is generated in the corresponding pull wires due to the curvature and shortening of portions of the connecting conduit 110 during flexing.

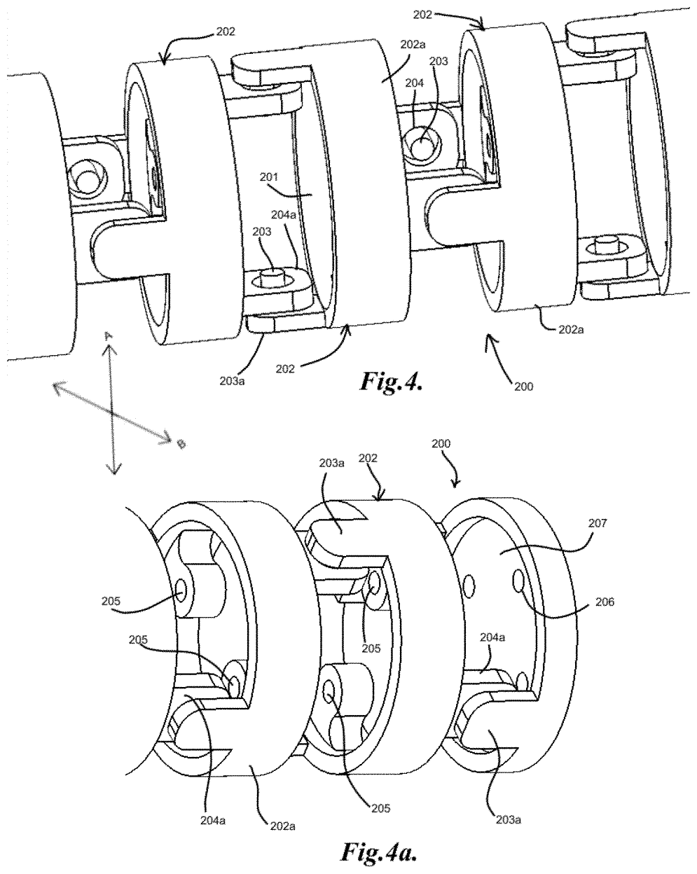

In some embodiments, a plurality of interconnecting segments may be utilized that connect and articulate relative to each other. FIG. 4 illustrates an example of interconnecting segments 202 forming a support structure 200 for connecting conduit 110 with a channel 201 along its length for carrying conduits or connections within, as discussed below. As illustrated, the interconnecting segments 202 may be substantially identical and in the form of a ring 202a and may link to each other via rivets 203 extending from rivet extensions 203a that rest in and freely rotate in node rings 204 extending from node extensions 204a. Further, the connections between successive interconnecting segments 202 may be offset, such as, for example, at 90 degrees as illustrated, such that two adjacent interconnecting segments 202 may pivot in one axis, and the successive pair may pivot in a different axis, such as with vertical pivot A and horizontal pivot B such that the support structure 200 may be steered in two dimensions by pulling and/or pushing an appropriate pull wire(s), which may rest in wire carriers 205 and attach or be anchored at a distal end plate, as shown with anchoring points 206 at end piece 207 of the distal portion of support structure 200 in FIG. 4b. Other variations, such as offsets of different angles and the addition or subtraction of pairs of pull wires may also be utilized. Further examples of interconnecting segments are disclosed in U.S. Patent Publication US20090209819, which is hereby incorporated by reference in its entirety.

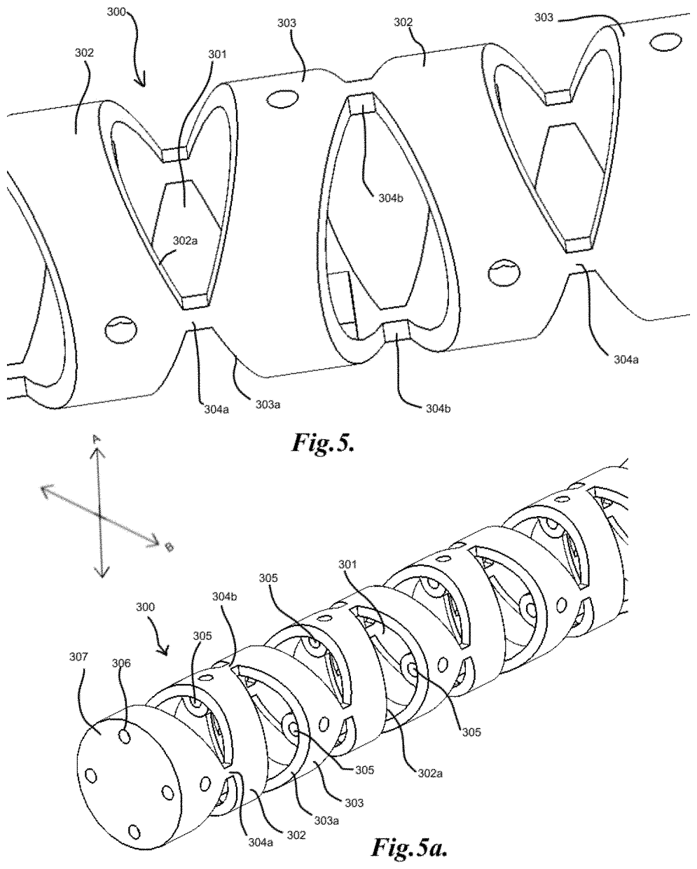

In other exemplary embodiments, the connecting conduit 110 may include a unitary flexible or deformable support structure which may interact with the pull wires to direct the distal assembly 120 of the connecting conduit 110 in a desired direction or orientation. FIGS. 5 and 5a illustrate an example of a unitary support structure 300 featuring a channel 301 with a series of offset ring-shaped sections with a first set 302 and a second set 303 offset at 90 degrees from first set 302. The first set 302 may generally connect to second set 303 with pairs of flexing bridges 304a, 304b, each pair of which may generally allow for flexion in at least one direction. The first set 302 and second set 303 may further include scalloped or other cutouts, such as scalloped cutouts 302a, 303a, which may generally form gaps in the structure and conform to each other when the unitary support structure 300 is flexed. Further, the connections at the bridges 304a, 304b between successive sets 302, 303, such as, for example, at 90 degrees as illustrated, may allow a pivot in one axis, and the successive sets may pivot in a different axis, such as with vertical pivot A and horizontal pivot B such that the support structure 300 may be steered in two dimensions by pulling and/or pushing an appropriate pull wire(s), which may rest in wire carriers 305 and attach or be anchored at a distal end plate, as shown with anchoring points 306 at end piece 307 of the distal portion of support structure 300 in FIG. 5a. Other variations, such as offsets of different angles and the addition or subtraction of pairs of pull wires may also be utilized.

The support structures, such as support structures 200, 300, may be made from any suitable material, such as polymers, metals, composites, and/or any other appropriate material or combinations thereof. For the unitary support structure 300 and similar embodiments, the material chosen may generally be flexible and durable against repeated flexions without failure. Suitable polymers may include, but are not limited to, polyethylene; polypropylene; polybutylene; polystyrene; polyester; polytetrafluoroethylene (PTFE); acrylic polymers; polyvinylchloride; Acetal polymers such as polyoxymethylene or Delrin (available from DuPont Company); natural or synthetic rubber; polyamide, or other high temperature polymers such as polyetherimide like ULTEM®, a polymeric alloy such as Xenoy® resin, which is a composite of polycarbonate and polybutyleneterephthalate, Lexan® plastic, which is a copolymer of polycarbonate and isophthalate terephthalate resorcinol resin (all available from GE Plastics); liquid crystal polymers, such as an aromatic polyester or an aromatic polyester amide containing, as a constituent, at least one compound selected from the group consisting of an aromatic hydroxycarboxylic acid (such as hydroxybenzoate (rigid monomer), hydroxynaphthoate (flexible monomer), an aromatic hydroxyamine and an aromatic diamine, (exemplified in U.S. Pat. Nos. 6,242,063, 6,274,242, 6,643,552 and 6,797,198, the contents of which are incorporated herein by reference), polyesterimide anhydrides with terminal anhydride group or lateral anhydrides (exemplified in U.S. Pat. No. 6,730,377, the content of which is incorporated herein by reference) or combinations thereof. Some of these materials are recyclable or may be made to be recyclable. Compostable or biodegradable materials may also be used and may include any biodegradable or biocompostable polyesters such as a polylactic acid resin (comprising L-lactic acid and D-lactic acid) and polyglycolic acid (PGA), polyhydroxyvalerate/hydroxybutyrate resin (PHBV) (copolymer of 3-hydroxy butyric acid and 3-hydroxy pentanoic acid (3-hydroxy valeric acid) and polyhydroxyalkanoate (PHA) copolymers, and polyester/urethane resin. Some non-compostable or non-biodegradable materials may also be made compostable or biodegradable by the addition of certain additives, for example, any oxo-biodegradable additive such as D2W™ supplied by (Symphony Environmental, Borehamwood, United Kingdom) and TDPA® manufactured by EPI Environmental Products Inc. Vancouver, British Columbia, Canada.

In addition, any polymeric composite such as engineering prepregs or composites, which are polymers filled with pigments, carbon particles, silica, glass fibers, or mixtures thereof may also be used. For example, a blend of polycarbonate and ABS (Acrylonitrile Butadiene Styrene) may be used for the housing. For further example, carbon-fiber and/or glass-fiber reinforced plastic may also be used.

Useful metals or metallic materials may include metal and metal alloys such as aluminum, steel, stainless steel, nickel titanium alloys, shape memory alloys and so on.

In some embodiments, the support structures 200, 300 may be supplemented to aid in preventing unwanted compression or to provide a return force, such as with a return spring. For example, when the support structures 200, 300 are bent or deformed during use, the supplementation may be utilized to return the support structures 200, 300 to their original states. For example, a conduit tube may be provided, such as a working channel, which may provide additional rigidity and/or act as a return spring. The conduit tube may be, for example, pinned or otherwise attached to the ends of the support structures 200, 300, as illustrated in FIG. 2c with pins 114, 115 pinning ends of the support structure 200/300 to a semi-rigid conduit tube 110b within the connecting conduit 110.

In some embodiments, the support structure 200 or 300 may be integral to the connecting conduit 110, such as by forming the support structure 200 or 300 by modification of the connecting conduit material or a portion thereof, such as by cutting or otherwise removing portions of the material to form the support structure 200 or 300. The connecting conduit 110 may further include an outer sheath to cover the modified portions.

In some exemplary embodiments, connections may be utilized to provide fluid/gas/vacuum supplies for fluid communication to the distal assembly, as illustrated in FIGS. 1, 1a, 1b. In general, for insertion and/or guiding the device 100 into a body cavity, such as a body tube, for example, the intestines and/or connecting structures, fluid, gas and or vacuum may be useful to aid in lubricating, opening and/or otherwise manipulating the body cavity for ease of access and/or directing of the device 100. As illustrated, fluid/gas/vacuum may be controlled with control valve 105, which may actuate valves for vacuum lines 108a, 108b, and control valve 106, which may actuate valves for water line 111a, air line 109a for feeding into fluid line 109b. The fluid/gas/vacuum may further be connected to act on the working channel aperture 121 of the distal assembly 120 to affect the body cavity.

The distal assembly 120 may also be adapted to ease access into the body, such as with rounded and/or contoured tip 124, as illustrated in FIG. 3. In general, the distal assembly 120 may also feature rounded and/or non-sharp features for minimizing and/or preventing damage to body tissues during use.

In another aspect, the device may generally include a camera in the distal assembly for visualizing body tissues. In some exemplary embodiments, the camera may be side viewing relative to the axis of the insertion of the device. FIG. 3 illustrates the distal assembly 120 of the device 100 with a side viewing camera 123, working channel aperture 121, elevator 122.

Further, the camera may generally be connected through the connecting conduit and/or handpiece for real time viewing and/or on demand visual capture during a procedure. The camera and/or processing system for the camera may also include, for example, dynamic and/or directional brightness control, such as with a light sensor, panoramic image capture/image stitching, image stabilization and/or other features.

Devices may also utilize modular designs which may include independently replaceable portions, such as disposable portions and reusable portions. Portions may also be designed to be hot-swappable, such as to accommodate replacement of portions, such as due to malfunction or the like, during use. For example, portions of the device 100 may be separable from each other to enable swapping of components and/or disposal of certain portions. In some embodiments, the handpiece 100a, connecting conduit 110, the distal assembly 120, and/or the controls 101, 102, 103, 104 may be separate pieces which may be replaced independently.

Although the invention has been described with respect to specific embodiments thereof, these embodiments are merely illustrative, and not restrictive of the invention. The description herein of illustrated embodiments of the invention, including the description in the Abstract and Summary, is not intended to be exhaustive or to limit the invention to the precise forms disclosed herein (and in particular, the inclusion of any particular embodiment, feature or function within the Abstract or Summary is not intended to limit the scope of the invention to such embodiment, feature or function). Rather, the description is intended to describe illustrative embodiments, features and functions in order to provide a person of ordinary skill in the art context to understand the invention without limiting the invention to any particularly described embodiment, feature or function, including any such embodiment feature or function described in the Abstract or Summary. While specific embodiments of, and examples for, the invention are described herein for illustrative purposes only, various equivalent modifications are possible within the spirit and scope of the invention, as those skilled in the relevant art will recognize and appreciate. As indicated, these modifications may be made to the invention in light of the foregoing description of illustrated embodiments of the invention and are to be included within the spirit and scope of the invention. Thus, while the invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the invention.

Reference throughout this specification to “one embodiment”, “an embodiment”, or “a specific embodiment” or similar terminology means that a particular feature, structure, or characteristic described in connection with the embodiment is included in at least one embodiment and may not necessarily be present in all embodiments. Thus, respective appearances of the phrases “in one embodiment”, “in an embodiment”, or “in a specific embodiment” or similar terminology in various places throughout this specification are not necessarily referring to the same embodiment. Furthermore, the particular features, structures, or characteristics of any particular embodiment may be combined in any suitable manner with one or more other embodiments. It is to be understood that other variations and modifications of the embodiments described and illustrated herein are possible in light of the teachings herein and are to be considered as part of the spirit and scope of the invention.

In the description herein, numerous specific details are provided, such as examples of components and/or methods, to provide a thorough understanding of embodiments of the invention. One skilled in the relevant art will recognize, however, that an embodiment may be able to be practiced without one or more of the specific details, or with other apparatus, systems, assemblies, methods, components, materials, parts, and/or the like. In other instances, well-known structures, components, systems, materials, or operations are not specifically shown or described in detail to avoid obscuring aspects of embodiments of the invention. While the invention may be illustrated by using a particular embodiment, this is not and does not limit the invention to any particular embodiment and a person of ordinary skill in the art will recognize that additional embodiments are readily understandable and are a part of this invention.

As used herein, the terms “comprises,” “comprising,” “includes,” “including,” “has,” “having,” or any other variation thereof, are intended to cover a non-exclusive inclusion. For example, a process, product, article, or apparatus that comprises a list of elements is not necessarily limited only those elements but may include other elements not expressly listed or inherent to such process, process, article, or apparatus.

Furthermore, the term “or” as used herein is generally intended to mean “and/or” unless otherwise indicated. For example, a condition A or B is satisfied by any one of the following: A is true (or present) and B is false (or not present), A is false (or not present) and B is true (or present), and both A and B are true (or present). As used herein, including the claims that follow, a term preceded by “a” or “an” (and “the” when antecedent basis is “a” or “an”) includes both singular and plural of such term, unless clearly indicated within the claim otherwise (i.e., that the reference “a” or “an” clearly indicates only the singular or only the plural). Also, as used in the description herein, the meaning of “in” includes “in” and “on” unless the context clearly dictates otherwise.

1. A device for imaging a body cavity comprising:
a handpiece;
a connecting conduit extending from said handpiece from a proximal end;
a distal assembly connected to a distal end of said connecting conduit;
a plurality of controls coupled to said handpiece connected to said distal assembly through a plurality of pull wires extending from mechanical actuators in said handpiece through said connecting conduit to said distal assembly, said plurality of pull wires being adapted to alter the trajectory of said distal assembly;
a working channel having an entry point on said handpiece and extending through said connecting conduit to an aperture on said distal assembly; and
at least one sensor disposed on said distal assembly in communication with a sensor port on said handpiece.

2. A device for imaging a body cavity comprising:
a handpiece;
a connecting conduit extending from said handpiece from a proximal end;
a distal assembly connected to a distal end of said connecting conduit;
a plurality of controls digitally coupled to powered actuators in said handpiece connected to said distal assembly through a plurality of pull wires extending from said handpiece through said connecting conduit to said distal assembly, said plurality of pull wires being adapted to alter the trajectory of said distal assembly;
a working channel having an entry point on said handpiece and extending through said connecting conduit to an aperture on said distal assembly; and
at least one sensor disposed on said distal assembly in communication with a sensor port on said handpiece.

3. The device of claim 2, further comprising a plurality of fluid connections on said handpiece for supplying fluid, gas and/or vacuum to said aperture.

4. The device of claim 2, wherein at least one of said handpiece, connecting conduit, distal assembly and plurality of controls are modular sections which are removable and swappable.

5. The device of claim 2, wherein said at least one sensor is selected from the group consisting of a camera, digital camera, pH sensor, oxygen sensor, pressure sensor, accelerometer, position sensor, orientation sensor, temperature sensor, fluid or tissue analysis sensor, chemical composition sensor, imaging sensor and light sensor.

6. The device of claim 2, wherein said working channel is adapted to receive a medical device and convey it to said aperture.

7. The device of claim 2, further comprising an elevator and elevator actuator adapted to push on a medical device proximal to said aperture.

8. The device of claim 2, further comprising at least one additional working channel.

9. The device of claim 2, wherein said plurality of pull wires comprise at least a first set for altering the trajectory of said distal assembly up and down and a second set for altering the trajectory of said distal assembly left and right.

10. The device of claim 2, wherein said plurality of pull wires are constructed from a material selected from the group consisting of metal and metal alloys, carbon fibers, fiber glass, polymer strands, and natural fibers.

11. The device of claim 2, wherein said plurality of controls comprise a set of control wheels.

12. The device of claim 2, wherein said connecting conduit comprises a flexible section proximal to said distal assembly.

13. The device of claim 2, wherein said connecting conduit comprises a unitary support structure with a channel, a series of offset ring-shaped sections with a first set of sections and a second set of sections offset at approximately 90 degrees from said first set of sections, said first and second sets of sections being connected by pairs of flexing bridges which may generally allow for flexion in at least one direction.

14. The device of claim 1, wherein said mechanical actuators comprise rack and pinion actuators to impart linear motion onto said plurality of pull wires.

15. The device of claim 1, further comprising a locking mechanism and a locking control.

16. The device of claim 2, wherein said powered actuators comprise motors coupled to reels adapted to wind and unwind to impart linear motion onto said plurality of pull wires.

17. The device of claim 16, wherein said motors comprise a self-locking effect when unpowered.

18. The device of claim 16, further comprising position sensing features adapted to detect the position of the plurality of pull wires.

19. The device of claim 16, wherein pairs of said plurality of pull wires attach to said reels at attachment points spaced at 45 degrees apart on an arc or less.

20. The device of claim 2, wherein said plurality of controls couple digitally to said powered actuators through wired or wireless communication.

21-23. (canceled)
